(19) United States
(12) Patent Application Publication
(10) Pub. No.: US 2025/0125028 A1
(43) Pub. Date: Apr. 17, 2025

(54) INTERNET OF THINGS (IOT) BASED DRUG DELIVERY SYSTEM AND METHOD FOR IDENTIFYING EMERGENCY MEDICAL EVENTS AND PROVIDING RESCUE MEDICATION DURING THE EMERGENCY MEDICAL EVENTS

(71) Applicant: Auriva Health, Inc., New Hartford, NY (US)

(72) Inventors: Jolene Bressi, New Hartford, NY (US); Melinda Chu, Blue Ash, OH (US)

(21) Appl. No.: 18/918,171

(22) Filed: Oct. 17, 2024

Related U.S. Application Data
(60) Provisional application No. 63/590,791, filed on Oct. 17, 2023.

(51) Int. Cl. G16H 20/10; A61B 5/00; A61B 5/0205; A61B 5/145

(52) U.S. Cl. CPC G16H 20/10; A61B 5/02055; A61B 5/14532; A61B 5/14542; A61B 5/7282; A61B 5/74

(57) ABSTRACT

An internet of things (IoT) based drug delivery system for identifying emergency medical events and providing rescue medication during the emergency medical events of individuals, is provided. The IoT-based drug delivery system includes communication devices configured to obtain real-time health data associated with the individuals through user interfaces of the communication devices. The communication devices are configured to transmit the real-time health data associated with the individuals, to IoT-enabled drug delivery devices. The IoT-enabled drug delivery devices includes sensors configured to detect the real-time health data associated with the individuals, from the communication devices. The IoT-enabled drug delivery devices further includes processors configured to process the real-time health data, to detect the emergency medical events upon detecting the real-time health data from the sensors. The IoT-enabled drug delivery devices further includes drug delivery mechanisms configured to provide corresponding rescue medication responding to the emergency medical events.

### EARLIEST PRIORITY DATE

This application claims priority from a Provisional patent application filed in the United States of America having Patent Application No. 63/590,791, on Oct. 17, 2023, and titled “DEVICE FOR NON-INVASIVE EARLY IDENTIFICATION OF MEDICAL CONDITIONS AND INTERNET OF THINGS ENABLED DRUG DELIVERY”.

### TECHNICAL FIELD

Embodiments of the present disclosure relate to internet of things (IoT) based medical devices, and more particularly relate to an IoT-based drug delivery system for identifying one or more emergency medical events and providing rescue medication responding to the one or more emergency medical events of one or more individuals.

### BACKGROUND

In recent years, advancements in medical device technology and Internet of Things (IoT) integration have led to significant improvements in the field of healthcare. One notable trend is the development of non-invasive diagnostic devices and IoT-enabled drug delivery systems.

Non-invasive diagnostic devices have gained prominence due to their ability to provide valuable health information without the need for invasive procedures. These non-invasive diagnostic devices employ a range of technologies, including sensors, imaging, and biomarker analysis, to detect and monitor medical conditions. They have been instrumental in early disease detection, enabling more timely interventions and improved outcomes for individuals. For example, non-invasive diagnostic tools have been developed for conditions like diabetes, where continuous glucose monitoring systems allow individuals to track their blood sugar levels without requiring frequent fingerstick tests. In the field of cardiology, wearable devices equipped with ECG sensors may monitor heart rhythms and detect arrhythmias, offering early warning signs of potential cardiac issues.

Integrating IoT technology into healthcare devices has transformed how individuals and healthcare professionals manage and monitor medical conditions. IoT-enabled devices collect real-time data, transmit the collected data securely to cloud-based platforms, and enable remote monitoring and management of the health of individuals. For instance, IoT-enabled insulin pumps for diabetes management adjust insulin delivery based on real-time glucose levels and even send alerts to caregivers or healthcare providers in case of critical events. These IoT-enabled devices improve individual compliance and enable personalized treatment plans.

Additionally, IoT platforms are used to create interconnected ecosystems of healthcare devices, allowing for seamless data sharing and coordination among various medical devices and systems. This integration enhances the overall quality of care and supports remote individual monitoring, especially valuable for individuals with chronic conditions.

While non-invasive diagnostics and IoT-enabled drug delivery systems have shown promise, there are still challenges to overcome. Ensuring the accuracy and reliability of diagnostic data is critical, as is addressing privacy and security concerns in IoT-connected healthcare devices. Regulatory compliance and standardization efforts continue to evolve to ensure safety and effectiveness of these technologies.

In existing technology, an electronic nose development and preliminary human breath testing for rapid, non-invasive COVID-19 detection are disclosed. The electronic nose (E-Nose) is designed for rapid on-site screening of COVID-19 infection by analyzing volatile organic compounds (VOCs) in exhaled human breath. The E-Nose consists of a hand-held E-Nose sensor system with 64 chemically sensitive nanomaterial sensing elements tailored to detect COVID-19 VOCs. The E-Nose includes data acquisition electronics, a tablet with control software, and a sampling fixture to capture breath samples. However, the E-Nose may lack specificity, as the E-Nose relies on VOC patterns that are influenced by a plurality of factors, potentially leading to false positives or negatives. The system associated with the E-Nose is relatively complex, requiring multiple components and Nano-sensors. This complexity limits the ease of use and portability of the E-Nose compared to simpler non-invasive devices.

There are various technical problems with the non-invasive early identification of medical conditions and IoT-enabled drug delivery in the prior art. Many existing non-invasive diagnostic devices may lack the precision required for early identification of specific medical conditions. Variability in data accuracy and reliability may impact the effectiveness of diagnosis and treatment. Integration between different medical devices and platforms can be complex and problematic. Ensuring seamless data exchange and compatibility among various components in an IoT ecosystem is a technical hurdle. Detecting and treating serious or life-threatening events across a spectrum of medical conditions is a critical challenge in modern healthcare. Various conditions, including at least one of: seizures, myocardial infarction, diabetes-related hypoglycemia, sickle cell pain crises, cancer pain, opioid overdoses, benzodiazepine overdoses, and chemical exposures, require timely intervention to prevent adverse outcomes.

Therefore, there is a need for an IoT based drug delivery system for identifying one or more emergency medical events and providing rescue medication responding to the one or more emergency medical events of one or more individuals, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an internet of things (IoT) based drug delivery system for identifying one or more emergency medical events and providing rescue medication during the one or more emergency medical events of one or more individuals, is provided. The IoT-based drug delivery system comprises one or more communication devices configured to: (a) obtain real-time health data associated with the one or more individuals from the one or more individuals through one or more user interfaces of the one or more communication devices, and (b) transmit the real-time health data associated with the one or more individuals, to one or more internet of things (IoT) enabled drug delivery devices.

The IoT-based drug delivery system further comprises the one or more IoT-enabled drug delivery devices are communicatively connected to the one or more communication devices. The one or more IoT-enabled drug delivery devices comprise one or more sensors configured to detect the real-time health data associated with the one or more individuals, from the one or more communication devices. The one or more IoT-enabled drug delivery devices further comprise one or more processors configured to process the real-time health data associated with the one or more individuals, to detect the one or more emergency medical events upon detecting the real-time health data associated with the one or more individuals from the one or more sensors. The one or more IoT-enabled drug delivery devices further comprise one or more drug delivery mechanisms configured to provide a corresponding rescue medication responding to the one or more emergency medical events upon detecting the one or more emergency medical events.

In an embodiment, in processing the real-time health data associated with the one or more individuals, to detect the one or more emergency medical events, the one or more IoT-enabled drug delivery devices are configured to: (a) obtain the real-time health data associated with the one or more individuals, from the one or more sensors of the one or more IoT-enabled drug delivery devices, (b) compare the real-time health data associated with the one or more individuals, with one or more pre-stored health data associated with the one or more individuals, and (c) detect the one or more emergency medical events based on determination of one or more values pre-set for corresponding one or more emergency medical events, upon comparison of the real-time health data associated with the one or more individuals, with the one or more pre-stored health data associated with the one or more individuals.

In another embodiment, the one or more sensors comprise at least one of: (a) a continuous glucose monitoring sensor configured to detect glucose level in blood, (b) a breath sensor configured to detect at least one of: ethane, pentane, and relevant volatile organic compounds (VOC) corresponding to myocardial infarction, (c) a wearable electroencephalogram (EEG) sensor configured to detect one or more electrical activities of a brain, (d) an oxygen saturation sensor configured to detect blood oxygen level, and (e) an electrocardiogram sensor (ECG) configured to detect one or more electrical activities of a heart.

In yet another embodiment, in providing the corresponding rescue medication responding to the one or more emergency medical events upon detecting the one or more emergency medical events, the one or more IoT-enabled drug delivery devices are configured to at least one of: (a) automatically inject the corresponding rescue medication subcutaneously in response to the detection of the one or more emergency medical events occurred to the one or more individuals, using the one or more drug delivery mechanisms, and (b) inject the corresponding rescue medication subcutaneously by pressing one or more control buttons by at least one of: the one or more individuals and one or more users during the one or more emergency medical events, to operate the one or more drug delivery mechanisms. In an embodiment, one or more informative signals associated with the one or more emergency medical events are transmitted to the one or more communication devices to determine that the one or more individuals are having the one or more emergency medical events, when the one or more IoT-enabled drug delivery devices detect the one or more emergency medical events. The one or more informative signals transmitted to the one or more communication devices, allows the one or more users to press the one or more control buttons to inject the corresponding rescue medication subcutaneously to the one or more individuals during the one or more emergency medical events.

In yet another embodiment, the real-time health data associated with the one or more individuals comprise at least one of: one or more vital signs, one or more activity levels of at least one of: brain and heart, and one or more health metrics.

In yet another embodiment, the one or more communication devices are configured to: (a) generate one or more alert signals and notifications upon receiving the one or more informative signals associated with the one or more emergency medical events from the one or more IoT-enabled drug delivery devices, and (b) transmit the one or more alert signals and notifications to the one or more communications devices of at least one of: the one or more users and the one or more individuals, through the one or more user interfaces of the one or more communication devices.

In one aspect, an internet of things (IoT) based drug delivery method for identifying one or more emergency medical events and providing rescue medication during the one or more emergency medical events of one or more individuals, is disclosed. The IoT-based drug delivery method comprises obtaining, by one or more communication devices, real-time health data associated with the one or more individuals from the one or more individuals through one or more user interfaces of the one or more communication devices.

The IoT-based drug delivery method further comprises transmitting, by the one or more communication devices, the real-time health data associated with the one or more individuals, to one or more internet of things (IoT) enabled drug delivery devices. The IoT-based drug delivery method further comprises detecting, by one or more sensors of the one or more IoT-enabled drug delivery devices, the real-time health data associated with the one or more individuals, from the one or more communication devices.

The IoT-based drug delivery method further comprises processing, by one or more processors of the one or more IoT-enabled drug delivery devices, the real-time health data associated with the one or more individuals, to detect the one or more emergency medical events upon detecting the real-time health data associated with the one or more individuals from the one or more sensors. The IoT-based drug delivery method further comprises providing, by one or more drug delivery mechanisms of the one or more IoT-enabled drug delivery devices, a corresponding rescue medication responding to the one or more emergency medical events upon detecting the one or more emergency medical events.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating a computing environment with an internet of things (IoT) based drug delivery system for identifying one or more emergency medical events and providing rescue medication responding to the one or more emergency medical events of one or more individuals, in accordance with an embodiment of the present disclosure;

FIG. 2 is a detailed view of the IoT-based drug delivery system for identifying the one or more emergency medical events and providing the rescue medication responding to the one or more emergency medical events of the one or more individuals, in accordance with an embodiment of the present disclosure;

FIG. 3 illustrates an exemplary IoT-enabled drug delivery device with a portable design, in accordance with an embodiment of the present disclosure;

FIG. 4 illustrates an exemplary IoT-enabled drug delivery device with a wearable design, in accordance with an embodiment of the present disclosure;

FIGS. 5A-5C illustrate exemplary diverse designs of IoT-enabled drug delivery devices, in accordance with an embodiment of the present disclosure; and

FIG. 6 illustrates a flow chart illustrating an IoT-based drug delivery method for identifying the one or more emergency medical events and providing the rescue medication responding to the one or more emergency medical events of the one or more individuals, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

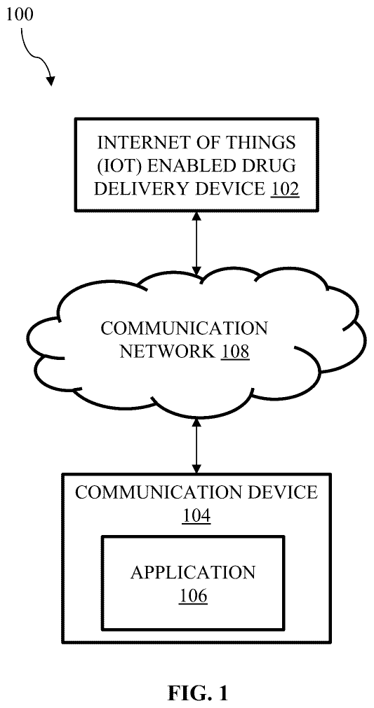

FIG. 1 is a block diagram illustrating a computing environment with an internet of things (IoT) based drug delivery system 100 for identifying one or more emergency medical events and providing rescue medication responding to the one or more emergency medical events of one or more individuals, in accordance with an embodiment of the present disclosure.

As shown in FIG. 1, the IoT-based drug delivery system 100 includes one or more IoT-enabled drug delivery devices 102 works in conjunction with one or more communication devices 104 and a mobile application 106. The one or more IoT-enabled drug delivery devices 102 are designed to respond to the one or more emergency medical events autonomously and rapidly by utilizing the one or more communication devices 104. In an embodiment, the terms “one or more emergency medical events and “one or more critical medical events are used interchangeably. The one or more communication devices 104 are configured to obtain real-time health data associated with the one or more individuals from the one or more individuals through one or more user interfaces (e.g., the mobile application 106). When the one or more communication devices 104 detect/obtain the real-time health data, the one or more communication devices 104 are configured to transmit the real-time health data associated with the one or more individuals, to the IoT-enabled drug delivery devices 102 through a communication network 108.

In other words, when the one or more communication devices 104 detect/obtain the real-time health data, the one or more communication devices 104 are configured to transmit one or more signals to the one or more IoT-enabled drug delivery devices 102 through the communication network 108.

The one or more IoT-enabled drug delivery devices 102 are communicatively connected to the one or more communication devices 104. The one or more communication devices 104 may be digital devices, computing devices and/or networks. The one or more communication devices 104 may include, but is not limited to, a mobile device, a smartphone, a personal digital assistant (PDA), a tablet computer, a phablet computer, a wearable computing device, a laptop, a desktop, a server, and the like. The communication network 108 may be a wired communication network and/or a wireless communication network, a local area network (LAN), a wide area network (WAN), a metropolitan area network (MAN), a telephone network, such as the public switched telephone network (PSTN) or a cellular network, an intranet, an internet, a fiber optic network, a satellite network, a cloud computing network, or a combination of networks.

The one or more IoT-enabled drug delivery devices 102 are configured to detect the real-time health data associated with the one or more individuals, obtained from the one or more communication devices 104. The one or more IoT-enabled drug delivery devices 102 are further configured to process the real-time health data associated with the one or more individuals, to detect the one or more emergency medical events upon detecting the real-time health data associated with the one or more individuals from the one or more sensors. The one or more IoT-enabled drug delivery devices 102 are further configured to provide a corresponding rescue medication responding to the one or more emergency medical events upon detecting the one or more emergency medical events. In an embodiment, the one or more emergency medical events may include but not limited to seizures, myocardial infarction, diabetes-related hypoglycemia, sickle cell pain crises, cancer pain, opioid overdoses, benzodiazepine overdoses, chemical exposures, and the like.

In an exemplary embodiment, the mobile application 106 is configured to be accessible to one or more users on the one or more communication devices 104. Integration of the mobile application 106 with the one or more IoT-enabled drug delivery devices 102 enhances the overall user experience, making the system easier for the one or more users to manage their health, and receive timely assistance during the one or more emergency medical events. The mobile application 106 is adapted to facilitate data visualization and display the real-time health data collected by the one or more communication devices 104. The real-time health data includes, but not limited to, one or more vital signs, one or more activity levels, any relevant health metrics, and the like, on the one or more communication devices 104.

In an embodiment, when the one or more IoT-enabled drug delivery devices 102 detect the one or more emergency medical events, the one or more communication devices 104 are configured to generate one or more alert signals and notifications on the mobile application 106 upon receiving the one or more informative signals associated with the one or more emergency medical events from the one or more IoT-enabled drug delivery devices 102. In an embodiment, the one or more communication devices 104 are configured to transmit the one or more alert signals and notifications to the one or more communications devices 104 of at least one of: the one or more users and the one or more individuals, through the one or more user interfaces (e.g., the mobile application 106) of the one or more communication devices 104. The one or more alert signals and notifications may include, but not limited to, visual, audible, haptic, and the like, ensuring that the one or more individuals are promptly informed of the emergency medical events. The caregiver or the family members may also install the mobile application 106 to remotely monitor the real-time health data of the individual, thereby enhancing the support network for the individual.

During operation, when the one or more individuals experience the one or more emergency medical events, the one or more IoT-enabled drug delivery devices 102 respond to the one or more emergency medical events rapidly by means of the one or more communication devices 104. On detecting the one or more emergency medical events, the prompt signal is transmitted to the one or more IoT-enabled drug delivery devices 102 to initiate the delivery of the rescue medication. The prompt signal transmitted to the one or more IoT-enabled drug delivery devices 102 includes, but not limited to, at least one of: Bluetooth, Near-Field Communication (NFC), ZigBee, Infrared (IR), Wi-Fi (wireless fidelity), and the like.

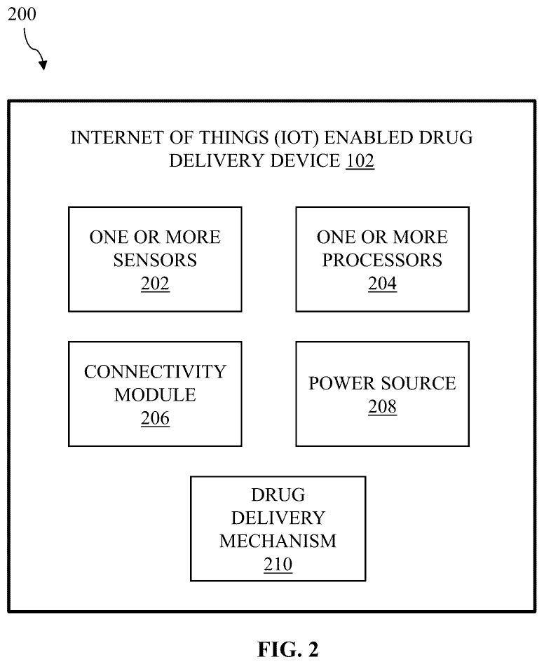

FIG. 2 is a detailed view 200 of the one or more IoT-based drug delivery devices 102 for identifying the one or more emergency medical events and providing the rescue medication responding to the one or more emergency medical events of the one or more individuals, in accordance with an embodiment of the present disclosure. The one or more IoT-based drug delivery devices 102 may include one or more sensors 202, one or more processors 204 (i.e., one or more hardware processors 204), a connectivity module 206, a power source 208, and one or more drug delivery mechanisms 210.

The one or more sensors 202 are configured to detect the real-time health data associated with the one or more individuals, from the one or more communication devices 104. The one or more sensors 202 may include at least one of: a continuous glucose monitoring sensor, a breath sensor, a wearable electroencephalogram (EEG) sensor, an oxygen saturation sensor, an electrocardiogram sensor, and the like. The continuous glucose monitoring sensor is configured to detect glucose level in blood. The breath sensor is configured to detect at least one of: ethane, pentane, and relevant volatile organic compounds (VOC) corresponding to myocardial infarction. The wearable electroencephalogram (EEG) sensor is configured to detect one or more electrical activities of a brain. The oxygen saturation sensor is configured to detect blood oxygen level. The electrocardiogram sensor (ECG) is configured to detect one or more electrical activities of a heart. Additionally, one or more wearable glasses tracking eye movements may also play a role in detecting conditions like seizures or neurological issues. In an embodiment, each of the one or more sensors/devices may use its own detection algorithms (e.g., AI, threshold comparisons) to continuously monitor real-time physiological health data including at least one of: glucose levels, brainwave patterns, VOCs, heart rhythms, and the like. When the emergency critical event is detected, these devices may communicate with the auto-injector of the one or more IoT-based drug delivery devices 102 to initiate the appropriate response, such as administering medication.

The one or more processors 204 are configured to process the real-time health data associated with the one or more individuals, to detect the one or more emergency medical events upon detecting the real-time health data associated with the one or more individuals from the one or more sensors 202. In other words, the one or more processors 204 are responsible for processing the real-time health data from the one or more sensors 202, executing algorithms for diagnosis, and managing functions of the one or more IoT-enabled drug delivery devices 102. The one or more processors 204 are configured to perform decision-making and communication with other components in the one or more IoT-enabled drug delivery devices 102.

For processing the real-time health data associated with the one or more individuals, to detect the one or more emergency medical events, the one or more processors 204 of the one or more IoT-enabled drug delivery devices 102 are initially configured to obtain the real-time health data associated with the one or more individuals, from the one or more sensors 202 of the one or more IoT-enabled drug delivery devices 102. The one or more processors 204 of the one or more IoT-enabled drug delivery devices 102 are further configured to compare the real-time health data associated with the one or more individuals, with one or more pre-stored health data associated with the one or more individuals. The one or more processors 204 of the one or more IoT-enabled drug delivery devices 102 are further configured to detect the one or more emergency medical events based on determination of one or more values pre-set for corresponding one or more emergency medical events, upon comparison of the real-time health data associated with the one or more individuals, with the one or more pre-stored health data associated with the one or more individuals.

The connectivity module 206 is configured to transmit data associated with the one or more emergency medical events, to the one or more communication devices 104 through the communication network 108. The connectivity module 206 is configured to provide real-time data transmission. The power source 208 in the one or more IoT-enabled drug delivery devices 102 may be a rechargeable battery or a replaceable one, depending on the intended usage duration of the one or more IoT-enabled drug delivery devices 102. Energy-efficient power management is crucial, especially for the one or more IoT-enabled drug delivery devices 102 with wearable design. The one or more drug delivery mechanisms 210 provides the corresponding rescue medication responding to the one or more emergency medical events, upon detecting the one or more emergency medical events. The one or more drug delivery mechanisms 210 may include one or more components including at least one of: pumps, needles, and the like. The one or more drug delivery mechanisms 210 is reliable, precise, and safe for subcutaneous, intramuscular, or other forms of medication delivery.

In an embodiment, the one or more drug delivery mechanisms 210 may be dual drug delivery mechanism (e.g., automatic mode and manual mode) in the one or more IoT-enabled drug delivery devices 102, based on the preference of the one or more individual and the severity of the one or more emergency medical events. In an embodiment, the one or more drug delivery mechanisms 210 are configured to automatically inject the corresponding rescue medication subcutaneously in response to the detection of the one or more emergency medical events occurred to the one or more individuals. When the one or more emergency medical event occurs, the one or more sensors 202 within the one or more IoT-enabled drug delivery devices 102 detect the events and trigger signals. The one or more IoT-enabled drug delivery devices 102, equipped with the necessary algorithms and mechanisms, are designed to receive, and process these signals. Upon receiving the signal indicating the one or more emergency medical events, the one or more IoT-enabled drug delivery devices 102 initiate the necessary response, such as administering the rescue medication automatically.

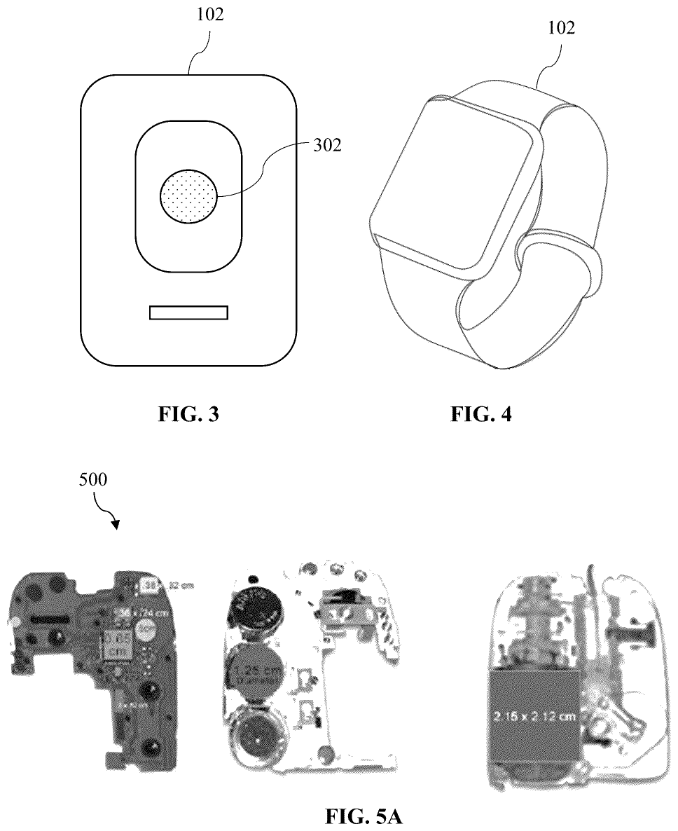

In another embodiment, the one or more IoT-enabled drug delivery devices 102 are configured to inject the corresponding rescue medication subcutaneously by pressing one or more control buttons (as shown in FIG. 3) by at least one of: the one or more individuals and one or more users during the one or more emergency medical events, to operate the one or more drug delivery mechanisms 210. One or more informative signals triggered by the one or more IoT-enabled drug delivery devices 102 are transmitted to the one or more communication devices 104 to ensure that the individual is promptly informed of the imminent emergency medical event. In such a case, the one or more control buttons are adapted to be pressed by the one or more users during the emergency medical event, thereby administering the rescue medication to the individual subcutaneously. The one or more IoT-enabled drug delivery devices 102 are thus tailored to assist in medical interventions across a range of emergency medical events.

FIG. 3 illustrates an exemplary IoT-enabled drug delivery device with a portable design, in accordance with an embodiment of the present disclosure.

In an exemplary embodiment, the one or more IoT-enabled drug delivery devices 102 are specifically designed to be compact, lightweight, and easily carried by the one or more individuals. The portable nature of the one or more IoT-enabled drug delivery devices 102 allow the one or more individuals to have the one or more IoT-enabled drug delivery devices 102 readily accessible and deployable in the one or more critical medical situations, ensuring that lifesaving medications are administered promptly, regardless of the location of the one or more individuals. The mobility and ease of use make the one or more IoT-enabled drug delivery devices 102 a valuable tool for managing and responding to various emergency medical conditions, even in non-clinical settings, where immediate intervention is essential. The one or more IoT-enabled drug delivery devices 102 are configured with the one or more control buttons 302 to manually operate the one or more IoT-enabled drug delivery devices 102 in the one or more critical medical events.

FIG. 4 illustrates an exemplary IoT-enabled drug delivery device with a wearable design, in accordance with an embodiment of the present disclosure.

In an exemplary embodiment, the one or more IoT-enabled drug delivery devices 102 with the wearable design is crafted to be worn comfortably by at least one of: the one or more users and the one or more individuals, allowing for continuous and unobtrusive monitoring and treatment during the one or more critical medical events. The wearable design ensures that the one or more individuals are able to have the one or more IoT-enabled drug delivery devices 102 on their body throughout daily activities, enabling rapid responses to the one or more critical medical events. The one or more IoT-enabled drug delivery devices 102 integration with wearables enhances individual autonomy and ensures that rescue medications are administered promptly, even before the arrival of emergency responders.

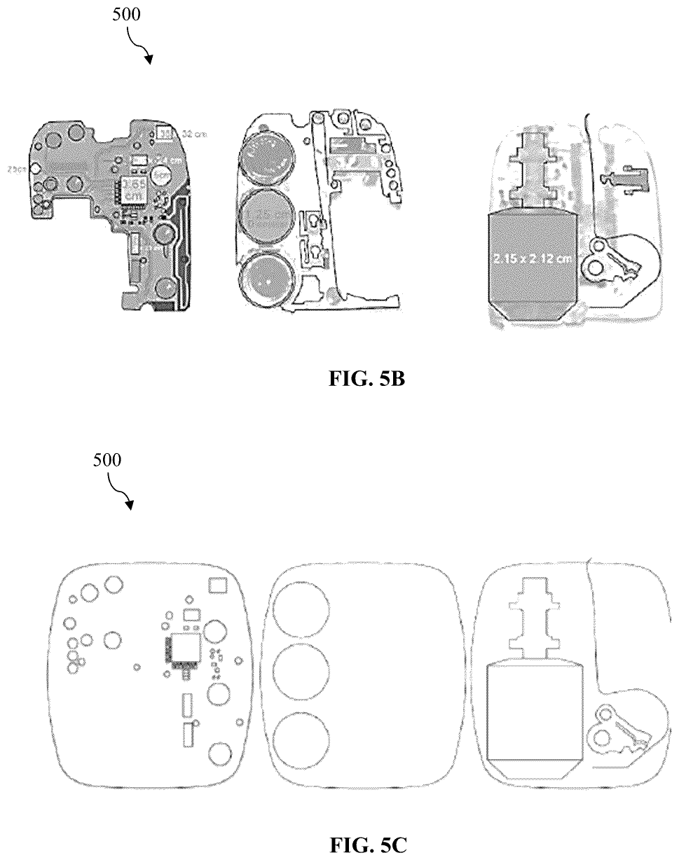

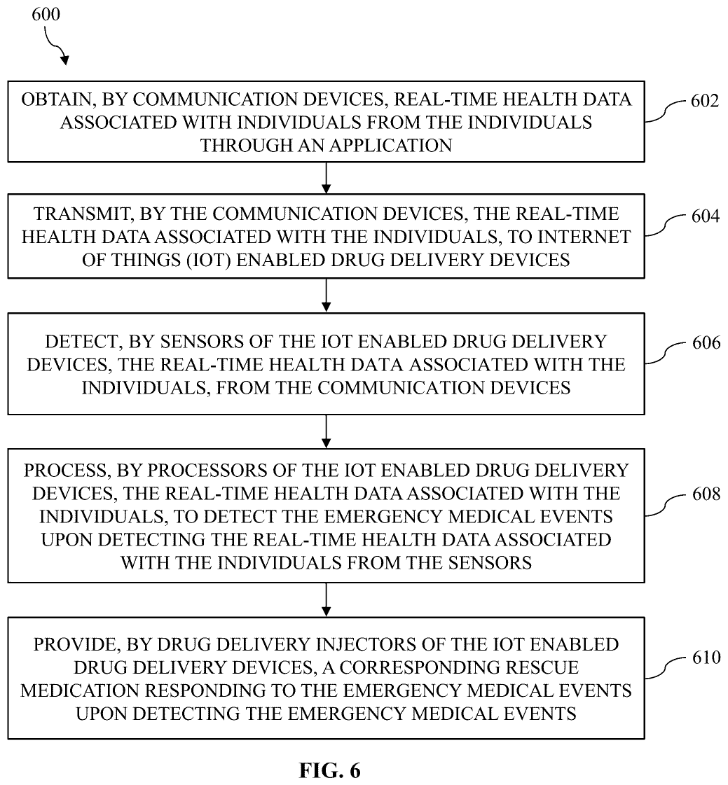

FIGS. 5A-5C illustrate exemplary diverse designs 500 of the one or more IoT-enabled drug delivery devices 102, in accordance with an embodiment of the present disclosure. FIGS. 5A-5C depict arrangements of one or more parts of the one or more IoT-enabled drug delivery devices 102, including at least one of: the one or more sensors 202, the one or more processors 204, the connectivity module 206, the power source 208 and the one or more drug delivery mechanisms 210. The detailed explanation for the above said parts have been described in FIG. 2.

In an exemplary embodiment, the one or more IoT-enabled drug delivery devices 102 are fabricated in diverse designs. The diverse designs cater to different user preferences and medical requirements. The range of options allows for customization and adaptation to various emergency medical conditions and individual needs. These designs may incorporate features including at least one of: ergonomics, form factor, and ease of use, ensuring that the one or more IoT-enabled drug delivery devices 102 are seamlessly integrated into daily lives of the one or more individuals while delivering the rescue medications in response to life-threatening events. This flexibility in design underscores the device's versatility and its potential to provide tailored solutions for a wide array of healthcare scenarios.

FIG. 6 illustrates a flow chart illustrating an IoT-based drug delivery method 600 for identifying the one or more emergency medical events and providing the rescue medication responding to the one or more emergency medical events of the one or more individuals, in accordance with an embodiment of the present disclosure.

At step 602, the real-time health data associated with the one or more individuals are obtained from the one or more individuals through one or more user interfaces (e.g., the mobile application 106) of the one or more communication devices 104.

At step 604, the real-time health data associated with the one or more individuals, are transmitted to one or more internet of things (IoT) enabled drug delivery devices 102.

At step 606, the real-time health data associated with the one or more individuals, are detected from the one or more communication devices 104.

At step 608, the real-time health data associated with the one or more individuals, are processed to detect the one or more emergency medical events upon detecting the real-time health data associated with the one or more individuals from the one or more sensors 202.

At step 610, the corresponding rescue medication responding to the one or more emergency medical events are provided to the one or more individuals upon detecting the one or more emergency medical events.

Numerous advantages of the present disclosure may be apparent from the discussion above. In accordance with the present disclosure, the one or more IoT-enabled drug delivery system 100 for early identification of the one or more emergency medical conditions and provision of the rescue medication responding to the one or more emergency medical events of one or more individuals, are disclosed. The one or more IoT-enabled drug delivery devices 102 enable the early detection of the one or more critical medical conditions, allowing for timely intervention and treatment initiation.

The one or more IoT-enabled drug delivery devices 102 facilitate personalized treatment plans by tailoring drug delivery to the individual's specific condition, ensuring the right medications are administered at the right time. Integration with IoT technology enables real-time data transmission, remote monitoring, and communication with healthcare providers, enhancing the quality of care and patient outcomes. The one or more IoT-enabled drug delivery devices 102 further facilitate an intuitive and user-friendly interface, making it accessible to the one or more individuals without extensive medical training.

The portable and wearable designs of the one or more IoT-enabled drug delivery devices 102 ensure that the one or more individuals are able to carry the one or more IoT-enabled drug delivery devices 102 with them wherever they go, allowing for continuous monitoring and immediate response in emergencies. By swiftly identifying the one or more critical medical events and initiating prompt treatment, the one or more IoT-enabled drug delivery devices 102 have the potential to save lives, reduce healthcare costs, and improve overall health outcomes.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the IoT-based drug delivery system 100 either directly or through intervening I/O controllers. Network adapters may also be coupled to the IoT-based drug delivery system 100 to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/IoT-based drug delivery system 100 in accordance with the embodiments herein. The IoT-based drug delivery system 100 herein comprises at least one processor or central processing unit (CPU). The CPUs are interconnected via a system bus to various devices including at least one of: a random-access memory (RAM), read-only memory (ROM), and an input/output (I/O) adapter. The I/O adapter can connect to peripheral devices, including at least one of: disk units and tape drives, or other program storage devices that are readable by the IoT-based drug delivery system 100. The IoT-based drug delivery system 100 can read the inventive instructions on the program storage devices and follow these instructions to execute the methodology of the embodiments herein.

The IoT-based drug delivery system 100 further includes a user interface adapter that connects a keyboard, mouse, speaker, microphone, and/or other user interface device including a touch screen device (not shown) to the bus to gather user input. Additionally, a communication adapter connects the bus to a data processing network, and a display adapter connects the bus to a display device which may be embodied as an output device including at least one of: a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that are issued on an application based here on. Accordingly, the embodiments of the present invention are intended to be illustrative, but not limiting, of the scope of the invention, which is set forth in the following claims.

What is claimed is:

1. An internet of things (IoT) based drug delivery system for identifying one or more emergency medical events and providing rescue medication during the one or more emergency medical events of one or more individuals, the IoT-based drug delivery system comprising:
one or more communication devices, wherein the one or more communication devices are configured to:
obtain real-time health data associated with the one or more individuals from the one or more individuals through one or more user interfaces of the one or more communication devices; and
transmit the real-time health data associated with the one or more individuals, to one or more internet of things (IoT) enabled drug delivery devices; and
the one or more IoT-enabled drug delivery devices are communicatively connected to the one or more communication devices, wherein the one or more IoT-enabled drug delivery devices comprise:
one or more sensors configured to detect the real-time health data associated with the one or more individuals, from the one or more communication devices;
one or more processors configured to process the real-time health data associated with the one or more individuals, to detect the one or more emergency medical events upon detecting the real-time health data associated with the one or more individuals from the one or more sensors; and
one or more drug delivery mechanisms configured to provide a corresponding rescue medication responding to the one or more emergency medical events upon detecting the one or more emergency medical events.

2. The IoT-based drug delivery system of claim 1, wherein in processing the real-time health data associated with the one or more individuals, to detect the one or more emergency medical events, the one or more IoT-enabled drug delivery devices are configured to:
obtain the real-time health data associated with the one or more individuals, from the one or more sensors of the one or more IoT-enabled drug delivery devices;
compare the real-time health data associated with the one or more individuals, with one or more pre-stored health data associated with the one or more individuals; and
detect the one or more emergency medical events based on determination of one or more values pre-set for corresponding one or more emergency medical events, upon comparison of the real-time health data associated with the one or more individuals, with the one or more pre-stored health data associated with the one or more individuals.

3. The IoT-based drug delivery system of claim 1, wherein the one or more sensors comprise at least one of:
a continuous glucose monitoring sensor configured to detect glucose level in blood;
a breath sensor configured to detect at least one of: ethane, pentane, and relevant volatile organic compounds (VOC) corresponding to myocardial infarction;
a wearable electroencephalogram (EEG) sensor configured to detect one or more electrical activities of a brain;
an oxygen saturation sensor configured to detect blood oxygen level; and
an electrocardiogram sensor (ECG) configured to detect one or more electrical activities of a heart.

4. The IoT-based drug delivery system of claim 1, wherein in providing the corresponding rescue medication responding to the one or more emergency medical events upon detecting the one or more emergency medical events, the one or more IoT-enabled drug delivery devices are configured to at least one of:
automatically inject the corresponding rescue medication subcutaneously in response to the detection of the one or more emergency medical events occurred to the one or more individuals, using the one or more drug delivery mechanisms; and
inject the corresponding rescue medication subcutaneously by pressing one or more control buttons by at least one of: the one or more individuals and one or more users during the one or more emergency medical events, to operate the one or more drug delivery mechanisms, wherein one or more informative signals associated with the one or more emergency medical events are transmitted to the one or more communication devices to determine that the one or more individuals are having the one or more emergency medical events, when the one or more IoT-enabled drug delivery devices detect the one or more emergency medical events, and
wherein the one or more informative signals transmitted to the one or more communication devices, allows the one or more users to press the one or more control buttons to inject the corresponding rescue medication subcutaneously to the one or more individuals during the one or more emergency medical events.

5. The IoT-based drug delivery system of claim 1, wherein the real-time health data associated with the one or more individuals comprise at least one of: one or more vital signs, one or more activity levels of at least one of: brain and heart, and one or more health metrics.

6. The IoT-based drug delivery system of claim 1, wherein the one or more communication devices are configured to:
generate one or more alert signals and notifications upon receiving the one or more informative signals associated with the one or more emergency medical events from the one or more IoT-enabled drug delivery devices; and
transmit the one or more alert signals and notifications to the one or more communications devices of at least one of: the one or more users and the one or more individuals, through the one or more user interfaces of the one or more communication devices.

7. An internet of things (IoT) based drug delivery method for identifying one or more emergency medical events and providing rescue medication during the one or more emergency medical events of one or more individuals, the IoT-based drug delivery method comprising:
obtaining, by one or more communication devices, real-time health data associated with the one or more individuals from the one or more individuals through one or more user interfaces of the one or more communication devices;
transmitting, by the one or more communication devices, the real-time health data associated with the one or more individuals, to one or more internet of things (IoT) enabled drug delivery devices;
detecting, by one or more sensors of the one or more IoT-enabled drug delivery devices, the real-time health data associated with the one or more individuals, from the one or more communication devices;
processing, by one or more processors of the one or more IoT-enabled drug delivery devices, the real-time health data associated with the one or more individuals, to detect the one or more emergency medical events upon detecting the real-time health data associated with the one or more individuals from the one or more sensors; and
providing, by one or more drug delivery mechanisms of the one or more IoT-enabled drug delivery devices, a corresponding rescue medication responding to the one or more emergency medical events upon detecting the one or more emergency medical events.

8. The IoT-based drug delivery method of claim 7, wherein processing the real-time health data associated with the one or more individuals, to detect the one or more emergency medical events, comprising:
obtaining, by the one or more processors of the one or more IoT-enabled drug delivery devices, the real-time health data associated with the one or more individuals, from the one or more sensors of the one or more IoT-enabled drug delivery devices;
comparing, by the one or more processors of the one or more IoT-enabled drug delivery devices, the real-time health data associated with the one or more individuals, with one or more pre-stored health data associated with the one or more individuals; and
detecting, by the one or more processors of the one or more IoT-enabled drug delivery devices, the one or more emergency medical events based on determination of one or more values pre-set for corresponding one or more emergency medical events, upon comparison of the real-time health data associated with the one or more individuals, with the one or more pre-stored health data associated with the one or more individuals.

9. The IoT-based drug delivery method of claim 7, wherein detecting the real-time health data associated with the one or more individuals, comprises:
detecting, by a continuous glucose monitoring sensor, glucose level in a blood;
detecting, by a breath sensor, at least one of: ethane, pentane, and relevant volatile organic compounds (VOC) corresponding to myocardial infarction;
detecting, by a wearable electroencephalogram (EEG) sensor, one or more electrical activities of a brain;
detecting, by an oxygen saturation sensor, blood oxygen level; and
detecting, by an electrocardiogram (ECG) sensor, one or more electrical activities of a heart.

10. The IoT-based drug delivery method of claim 7, wherein providing the corresponding rescue medication responding to the one or more emergency medical events upon detecting the one or more emergency medical events, comprises:
automatically injecting, by the one or more drug delivery mechanisms, the corresponding rescue medication subcutaneously in response to the detection of the one or more emergency medical events occurred to the one or more individuals, using the one or more drug delivery mechanisms; and
injecting, by the one or more drug delivery mechanisms, the corresponding rescue medication subcutaneously by pressing one or more control buttons by at least one of: the one or more individuals and one or more users during the one or more emergency medical events, to operate the one or more drug delivery mechanisms, wherein one or more informative signals associated with the one or more emergency medical events are transmitted to the one or more communication devices to determine that the one or more individuals are having the one or more emergency medical events, when the one or more IoT-enabled drug delivery devices detect the one or more emergency medical events, and
wherein the one or more informative signals transmitted to the one or more communication devices, allows the one or more users to press the one or more control buttons to inject the corresponding rescue medication subcutaneously to the one or more individuals during the one or more emergency medical events.

11. The IoT-based drug delivery method of claim 7, wherein the real-time health data associated with the one or more individuals comprise at least one of: one or more vital signs, one or more activity levels of at least one of: brain and heart, and one or more health metrics.

12. The IoT-based drug delivery method of claim 7, further comprising:
generating, by the one or more communication devices, one or more alert signals and notifications upon receiving the one or more informative signals associated with the one or more emergency medical events from the one or more IoT-enabled drug delivery devices; and
transmitting, by the one or more communication devices, the one or more alert signals and notifications to the one or more communications devices of at least one of: the one or more users and the one or more individuals, through the one or more user interfaces of the one or more communication devices.

13. A non-transitory computer-readable storage medium having instructions stored therein that when executed by one or more hardware processors, cause the one or more hardware processors to execute operations of:
obtaining real-time health data associated with the one or more individuals from the one or more individuals through one or more user interfaces of the one or more communication devices;
transmitting the real-time health data associated with the one or more individuals, to one or more internet of things (IoT) enabled drug delivery devices;
detecting the real-time health data associated with the one or more individuals, from the one or more communication devices;
processing the real-time health data associated with the one or more individuals, to detect the one or more emergency medical events upon detecting the real-time health data associated with the one or more individuals from the one or more sensors; and
providing a corresponding rescue medication responding to the one or more emergency medical events upon detecting the one or more emergency medical events.

14. The non-transitory computer-readable storage medium of claim 13, wherein processing the real-time health data associated with the one or more individuals, to detect the one or more emergency medical events, comprising:
obtaining the real-time health data associated with the one or more individuals, from the one or more sensors of the one or more IoT-enabled drug delivery devices;
comparing the real-time health data associated with the one or more individuals, with one or more pre-stored health data associated with the one or more individuals; and
detecting the one or more emergency medical events based on determination of one or more values pre-set for corresponding one or more emergency medical events, upon comparison of the real-time health data associated with the one or more individuals, with the one or more pre-stored health data associated with the one or more individuals.

15. The non-transitory computer-readable storage medium of claim 13, wherein detecting the real-time health data associated with the one or more individuals, comprises:
detecting glucose level in a blood;
detecting at least one of: ethane, pentane, and relevant volatile organic compounds (VOC) corresponding to myocardial infarction;
detecting one or more electrical activities of a brain;
detecting blood oxygen level; and
detecting one or more electrical activities of a heart.

16. The non-transitory computer-readable storage medium of claim 13, wherein providing the corresponding rescue medication responding to the one or more emergency medical events upon detecting the one or more emergency medical events, comprises:
automatically injecting the corresponding rescue medication subcutaneously in response to the detection of the one or more emergency medical events occurred to the one or more individuals, using the one or more drug delivery mechanisms; and
injecting the corresponding rescue medication subcutaneously by pressing one or more control buttons by at least one of: the one or more individuals and one or more users during the one or more emergency medical events, to operate the one or more drug delivery mechanisms, wherein one or more informative signals associated with the one or more emergency medical events are transmitted to the one or more communication devices to determine that the one or more individuals are having the one or more emergency medical events, when the one or more IoT-enabled drug delivery devices detect the one or more emergency medical events, and
wherein the one or more informative signals transmitted to the one or more communication devices, allows the one or more users to press the one or more control buttons to inject the corresponding rescue medication subcutaneously to the one or more individuals during the one or more emergency medical events.

17. The non-transitory computer-readable storage medium of claim 13, wherein the real-time health data associated with the one or more individuals comprise at least one of: one or more vital signs, one or more activity levels of at least one of: brain and heart, and one or more health metrics.

18. The non-transitory computer-readable storage medium of claim 13, further comprising:
generating one or more alert signals and notifications upon receiving the one or more informative signals associated with the one or more emergency medical events from the one or more IoT-enabled drug delivery devices; and
transmitting the one or more alert signals and notifications to the one or more communications devices of at least one of: the one or more users and the one or more individuals, through the one or more user interfaces of the one or more communication devices.
